(19) United States
(12) Patent Application Publication
(10) Pub. No.: US 2021/0177906 A1
(43) Pub. Date: Jun. 17, 2021

(54) ADIPOSE TISSUE PARTICLE PROCESSING, TRANSFER AND STORAGE SYSTEM

(71) Applicant: Roger S. Hogue, Maple Grove, MN (US)

(72) Inventor: Roger S. Hogue, Maple Grove, MN (US)

(21) Appl. No.: 17/119,930

(22) Filed: Dec. 11, 2020

Related U.S. Application Data
(60) Provisional application No. 62/946,701, filed on Dec. 11, 2019.

(51) Int. Cl. A61K 35/28; A61K 35/44; A61L 27/36; A61L 27/38; A61L 27/54; C12N 5/0775; C12N 5/071

(52) U.S. Cl. CPC A61K 35/28; A61K 35/44; A61L 27/3604; A61L 27/3834; A61L 27/54; C12N 2506/1384; C12N 5/069; A61L 2300/414; A61L 2300/416; A61L 2300/426; A61L 2430/34; C12N 5/0667

(57) ABSTRACT

An adipose tissue particle processing system includes a container and a filter screen assembly. The filter screen assembly has a first open end configured to receive adipose tissue from a syringe, and a second closed end opposite to the first open end located in the interior of the container. The filter screen assembly further includes a screen portion between the first open end and the second closed end, the screen portion including a plurality of apertures having diameters selected for processing the adipose tissue received through the first open end into controlled fat aspirate particle sizes that are output through the plurality of apertures into the interior of the container.

### CROSS-REFERENCE TO RELATED APPLICATION(S)

The present application claims the benefit of U.S. Provisional Application No. 62/946,701 filed on Dec. 11, 2020, which is incorporated by reference herein in its entirety.

### BACKGROUND

The present invention relates to systems and methods for processing, transferring and storing adipose tissue, such as fat aspirate obtained by liposuction.

Adipose tissue, or body fat, is loose connective tissue composed mostly of adipocytes, such as fat cells, along with a vast array of regenerative cell populations, including adipose-derived stem cells or mesenchymal stem cells, which have tremendous potential benefits for human tissue regeneration.

In order to harvest adipose tissue or fat aspirate containing regenerative call populations such as adipocyte-derived stem cells, a minimally-invasive treatment that uses tumescent liposuction techniques to harvest fat tissue as lipoaspirate can be used. Additional processing steps are routinely used following the initial harvesting procedure (i.e., tumescent liposuction), including fat aspirate particle sizing (micro-fragmenting or micronizing), filtering (removal of sinuate, connective tissue strands, and coarse debris), separating and concentrating (via gravity decanting or centrifugation to separate, isolate and remove water, blood, and oil from viable fat aspirate particles) in order to create an autologous fat graft that can be used for injection or deployment during an autologous fat grafting (fat transfer) treatment for the purpose of aesthetic (cosmetic) and/or regenerative purposes. Autologous fat grafting and/or autologous regenerative treatments containing autologous fat aspirate particles are used for cosmetic and/or therapeutic rejuvenation, restoration, and repair of aging or degenerative tissues such as the skin, hair, face, body, breasts, cleavage, dorsum of hands, soft tissue, wounds, scars, musculoskeletal tissues, vocal chords, and genitalia.

Currently, several procedures exist for processing (sizing, filtering, separating, and concentrating) fat aspirate particles. One such procedure involves placing the fat aspirate inside a chamber having many small steel balls immersed in saline. The chamber is then shaken whereby the steel balls micro-fragment the fat aspirate while the saline cleans it. This procedure can result in pulverization and indiscriminate sizing of the fat particles due to the high variability in shaking the chamber. Other procedures entail passing the fat aspirate back-and-forth many times across a mesh-like surface or screen with a square-shaped pattern to micronize the particles by using luer-to-luer syringe transfer. This processing can severely mechanically traumatize the fat aspirate particles and destroy the adipocyte cells, as well as be time consuming and physically straining. As a result, there is a need for systems and methods that result in precision processing (sizing, filtering, separating and concentrating) and single-pass outer dimensional sizing of fat aspirate obtained by liposuction harvesting for cosmetic and/or regenerative purposes.

### SUMMARY

An adipose tissue particle processing system according to the present disclosure includes a container and a filter screen assembly at least partially extending into an interior of the container. The filter screen assembly has a first open end configured to receive adipose tissue from a syringe, and a second closed end opposite to the first open end located in the interior of the container. The filter screen assembly further includes a screen portion between the first open end and the second closed end, the screen portion including a plurality of apertures having diameters selected for processing the adipose tissue received through the first open end into controlled fat aspirate particle sizes that are output through the plurality of apertures into the interior of the container.

A method of processing adipose tissue particles according to the present disclosure includes coupling a syringe containing adipose tissue particle material to an adipose tissue particle processing system that extends at least partially into a container. The adipose tissue particle material is transferred from the syringe via a first open end of a filter screen assembly of the adipose tissue particle processing system into an interior of the filter screen assembly, with a second end of the filter screen opposite the first open end being closed. Fat aspirate particles in the adipose tissue material are forced to pass through apertures in a screen portion of the filter screen assembly into the interior of the container, with a size of the fat aspirate particles being based on a size of the apertures in the screen portion.

### BRIEF DESCRIPTION OF THE DRAWINGS

The drawings are intended to illustrate embodiments of, but not to limit, the present invention. In the drawings, like reference characters denote corresponding features consistently throughout similar embodiments.

FIGS. 1A and 1B illustrate an adipose tissue particle processing system according to an embodiment of the present invention.

FIGS. 2A and 2B illustrate the assembly of the adipose tissue particle processing system shown in FIGS. 1A and 1B.

FIG. 3 illustrates a filter screen assembly for use in an adipose tissue particle processing system according to an embodiment of the present invention.

FIGS. 4A and 4B illustrate a cap/bushing for use in an adipose tissue particle processing system according to an embodiment of the present invention.

FIG. 5 illustrates a transfer cannula that may be used with an adipose tissue particle processing system according to an embodiment of the present invention.

FIG. 6 illustrates a cannula cleaner that may be used with an adipose tissue particle processing system according to an embodiment of the present invention

### DETAILED DESCRIPTION

The present disclosure provides an adipose tissue particle processing system that allows a physician to micro-fragment and process adipose tissue into controlled fat aspirate particle sizes for use in autologous fat grafting and/or autologous regenerative treatments containing the autologous fat aspirate particles.

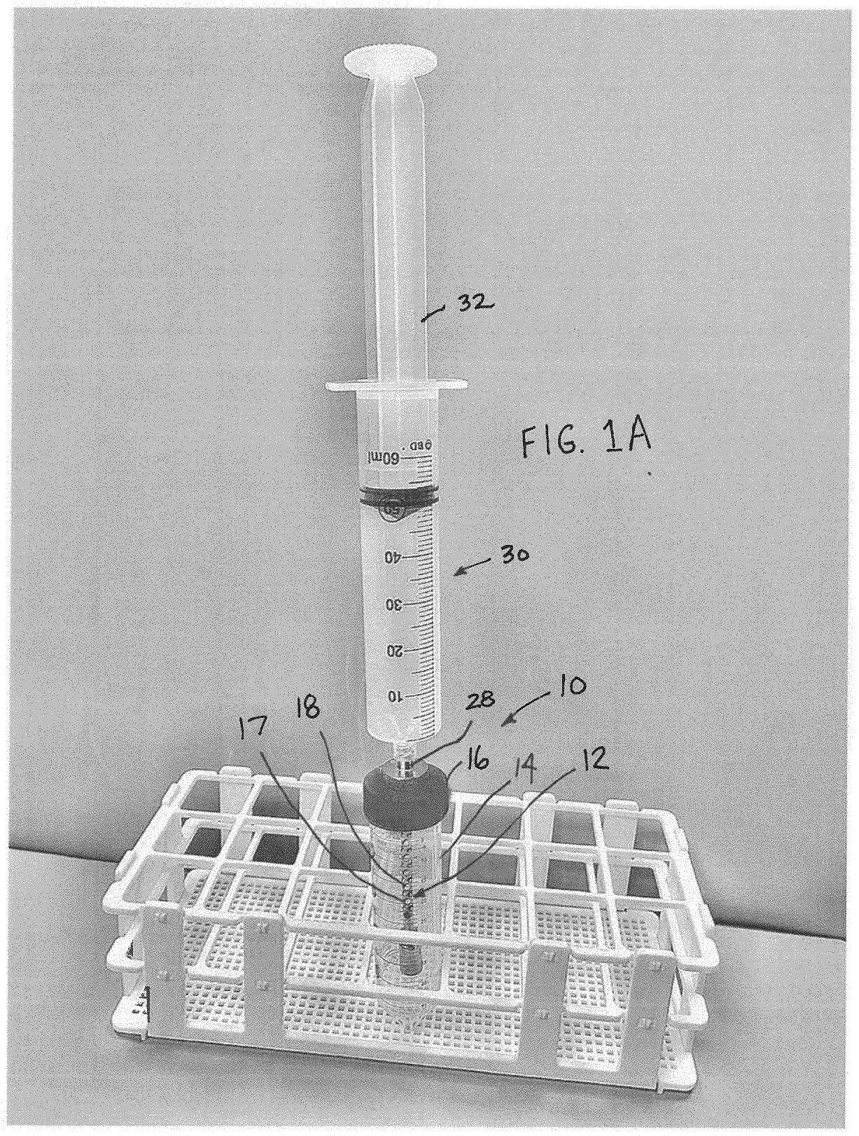

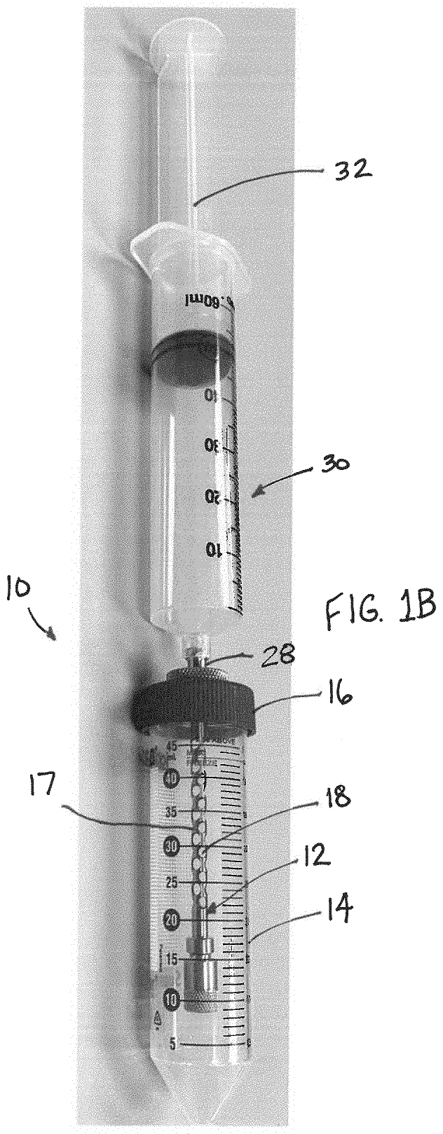

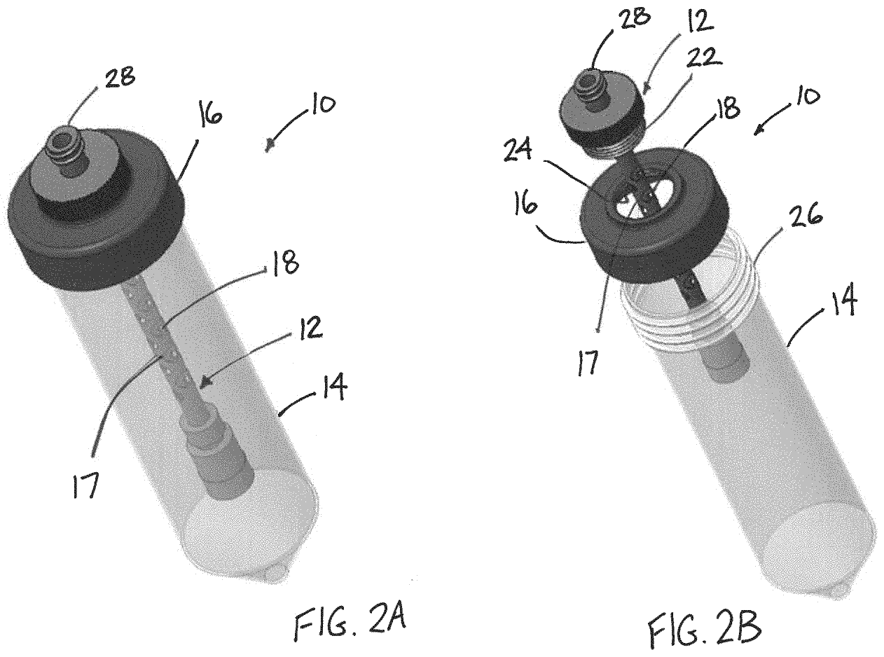

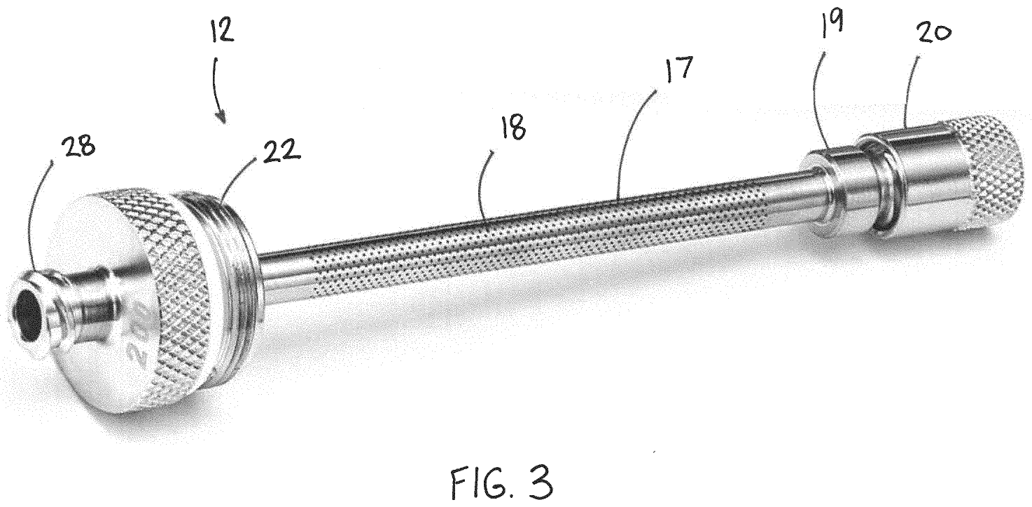

FIGS. 1A and 1B illustrate adipose tissue particle processing system 10 that includes a filter screen assembly 12 (which is also shown in more detail in FIG. 3) positioned to extend into plastic centrifuge tube 14 through cap/bushing 16. FIGS. 2A and 2B illustrate the assembly of the filter screen assembly 12 extending through cap/bushing 16 into the interior of centrifuge tube 14 of adipose tissue particle processing system 10. Filter screen assembly 12 includes screen portion 17 that is made up of a plurality of apertures 18 that have diameters selected for processing adipose tissue into controlled fat aspirate particle sizes. In the embodiment shown (see FIG. 3 in particular), the distal end of filter screen assembly 12 has female luer fitting 19, which allows male luer cap 20 to be attached to close the distal end of filter screen assembly 12 during use. Other methods or configurations for providing a closed distal end of filter screen assembly 12 during use may be used in alternative embodiments.

Also, in the embodiment shown (see FIGS. 2A and 2B in particular), filter screen assembly 12 includes male threads 22 near its proximal end, and cap/bushing 16 includes female threads 24 that are configured to receive male threads 22 of filter screen assembly 12, to secure filter screen assembly 12 to cap/bushing 16 so that screen portion 17 is suspended in the interior of centrifuge tube 14 when cap/bushing 16 is positioned on the top of centrifuge tube 14. In other embodiments, filter screen assembly 12 could alternatively be connected to a luer fitting or threaded fitting on cap/bushing 16, or could be integrally formed (e.g., by welding or adhesive connection) with cap/bushing 16.

In the embodiment shown, centrifuge tube 14 is made of clear plastic, and has a tapered configuration from its top (where cap/bushing 16 is provided) to its bottom (where a conical tapered end is provided). This is a common configuration for a plastic centrifuge tube, which is readily manufactured by injection molding, for example. In an alternative embodiment, a zero-draft, cylindrical plastic centrifuge tube may be constructed and used, which has no taper from the top to the bottom of the tube, and which has a flat bottom surface rather than a conical tapered end. With such a construction, the cylindrical plastic centrifuge tube could be used with the system described in U.S. patent application Ser. No. 16/295,695 entitled “Aspirating Separated Liquid Components From A Vessel” filed on Mar. 7, 2019, which is incorporated by reference herein in its entirety. In the system described in U.S. patent application Ser. No. 16/295,695, a diaphragm is slidably coupleable to the hollow inner portion of the centrifuge tube, and allows liquid contained in the centrifuge tube to be selectively and controllably aspirated out of the centrifuge tube through the diaphragm.

Centrifuge tube 14 shown in FIGS. 1A-2B is a 50 mL tube, but it should be understood that larger or smaller sizes and volumes of centrifuge tubes may be used in other embodiments.

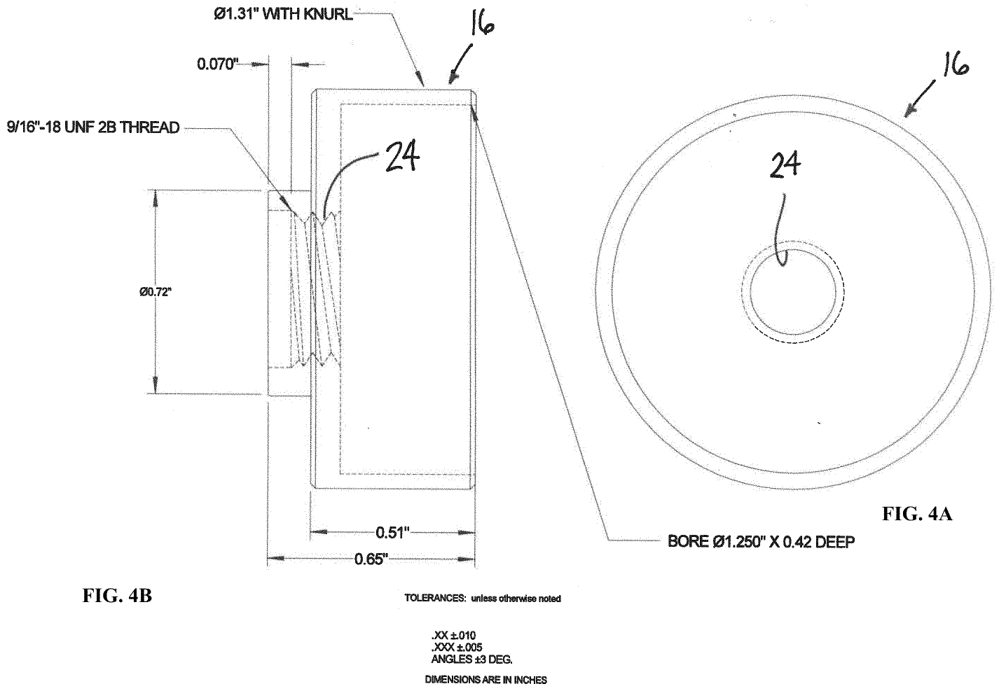

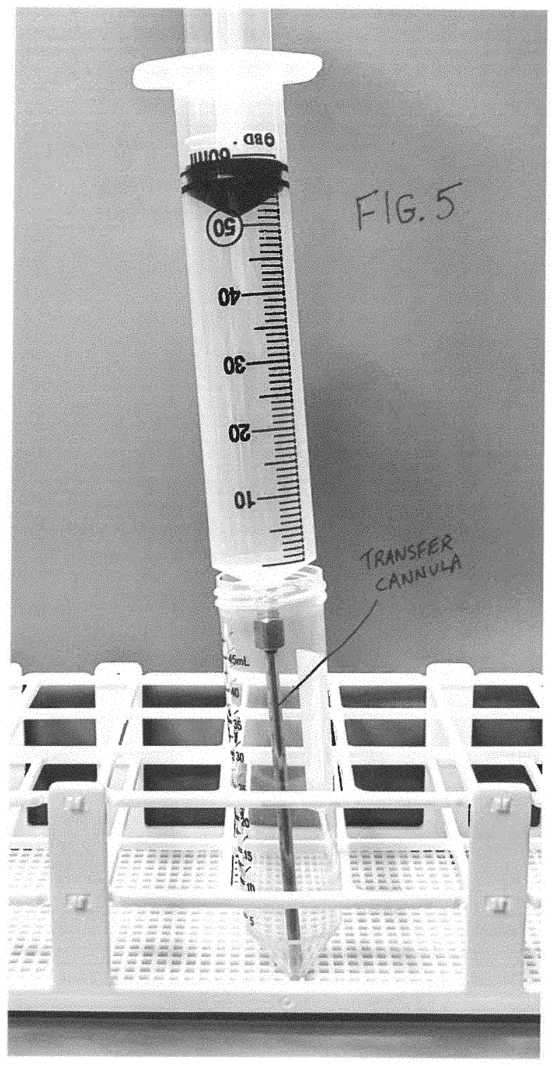

In the embodiment shown (see FIGS. 4A and 4B in particular), cap/bushing 16 is made of plastic, and has a threaded central aperture (having female threads 24) that engages with male threads 22 of filter screen assembly 12, so that screen portion 17 of filter screen assembly 12 is supported and suspended inside centrifuge tube 14. In the embodiment shown, cap 16/bushing is formed with a configuration that allows cap/bushing 16 to slip over male threads 26 at the top of centrifuge tube 14 (rather than threadedly engaging with male threads 26 at the top of centrifuge tube 14, as a standard lid for centrifuge tube 14 would do). With cap/bushing 16 configured to slip over male threads 26 at the top of centrifuge tube 14, venting is provided to allow depositing and aspirating of material to/from centrifuge tube 14, due to the non-airtight fitting between cap/bushing 16 and centrifuge tube 14. In alternative embodiments, cap/bushing 16 may have female threads which are threadedly engaged with male threads 26 at the top of centrifuge tube 14, thereby providing an airtight coupling between them, and cap/bushing 16 may be further designed to include venting apertures in its disc-shaped face, with a suitable air-permeable membrane, such as a 0.2 micron filter in some examples, to prevent liquid material from escaping through cap/bushing 16. In some alternative embodiments, cap/bushing 16 may be formed of stainless steel (with any of the variations of configurations described above), and may be a reusable component.

Exemplary dimensions for the various features of cap/bushing 16 are shown in FIGS. 4A and 4B. It should be understood that these dimensions are provided to illustrate one example of cap/bushing, and that the configuration of the features of cap/bushing 16 may have other dimensions either larger or smaller than the dimensions listed in other embodiments.

Apertures 18 in screen portion 17 of filter screen assembly 12 may be formed in by laser drilling in some embodiments. Example sizes/diameters of apertures 18 may be as large as 4.0 millimeters, as small as 0.2 millimeters, any size/diameter in between, or sizes/diameters larger than 4.0 millimeters or smaller than 0.2 millimeters, depending on the application in which the adipose tissue particle processing system 10 is used.

In one example, screen portion 17 of filter screen assembly 12 may have an outer diameter of about 0.259 inches (about 6.58 millimeters). In other examples, screen portion 17 of filter screen assembly 12 may have larger or smaller radial dimensions. In some embodiments, filter screen assembly 12 is composed of stainless steel.

In various embodiments, some of the components of adipose tissue particle sizing system 10 are designed to be reusable components (typically made of stainless steel), while other components are designed to be single-use, disposable components (typically made of plastic). In this context, components described as reusable are capable of being cleaned and sterilized multiple times, such as be a sterilizing autoclave, by enzyme treatment, or by other methods, while single-use, disposable components are provided in sterile packaging for a single use.

In operation, as shown in FIGS. 1A and 1B, female luer fitting 28 at the proximal end of filter screen assembly 12 is configured to allow coupling to the outlet of syringe 30, which can contain tissue material to be processed by adipose tissue particle processing system 10. Once syringe 30 is coupled to adipose tissue particle processing system 10, tissue material may be transferred into adipose tissue particle processing system 10 by pressing plunger 32 of syringe 30. This causes adipose tissue material to pass into the interior of screen portion 17 of filter screen assembly 12, with the distal end of filter screen assembly 12 being closed by luer cap 20, so that fat aspirate particles in the adipose tissue material are forced to pass from the interior of filter screen assembly 12 through apertures 18 of screen portion 17 into the interior of centrifuge tube 14. The fat aspirate particles are effectively “filtered” and “sized” (micro-fragmented) by sieve filtering and shearing force by apertures 18 of screen portion 17 of filter screen assembly 12, to a size that is determined by the size of apertures 18, while undesired sinuate, connective tissue strands, and coarse debris are not able to pass through apertures 18.

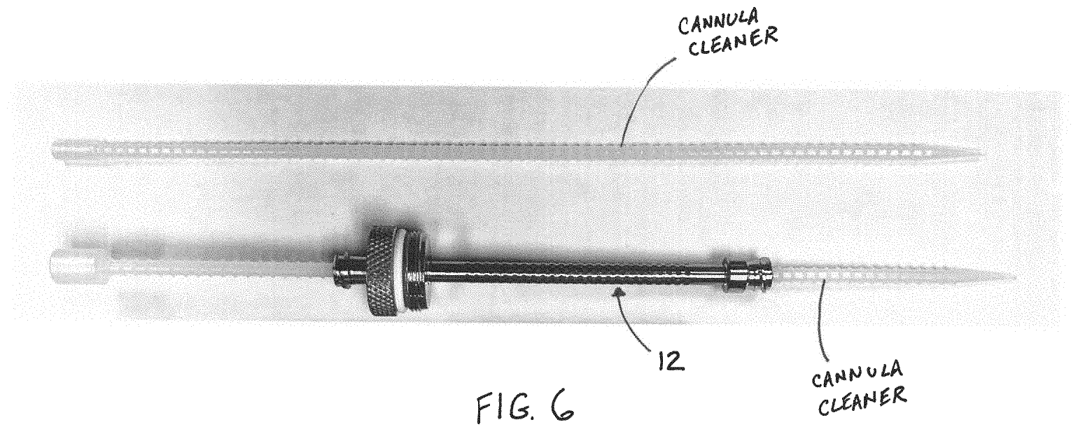

Once the micro-fragmented “sized” fat aspirate particles are transferred through screen portion 17 of filter screen assembly 12 into centrifuge tube 14, then centrifuge tube 14 may be prepared for centrifugation, by removing components of adipose tissue particle processing system 10, and replacing cap/bushing 16 with a conventional threaded lid. After the micro-fragmented fat aspirate particles are separated by either gravity decantation, or by centrifugation in a centrifuge system, various separated components may be aspirated from centrifuge tube 14. In some embodiments, aspiration may be performed by inserting a transfer cannula into the interior of centrifuge tube 14 and aspirating material through the transfer cannula with a syringe coupled to the transfer cannula (as illustrated in FIG. 5). The transfer cannula shown in FIG. 5 may be a 6-inch or 12-inch length cannula with a female luer-lock connector on its proximal end and an approximately 0.146-inch (3.7 mm) outer diameter cylindrical tubular blunt tip on its distal end In other embodiments, where centrifuge tube 14 has a zero-draft, cylindrical configuration, the method described in U.S. patent application Ser. No. 16/295,695 may be used, where a diaphragm is slidably coupleable to the hollow inner portion of centrifuge tube 14, and allows liquid contained in centrifuge tube 14 to be selectively and controllably aspirated out of centrifuge tube 14 through the diaphragm.

Filter screen assembly 12 may be cleaned after use by removing male luer cap 20 from the distal end, and inserting a cannula cleaner that is configured with projecting surfaces such as convex fins into the interior of filter screen assembly 12. Cleaning is performed by scraping, dislodging, and removing debris and contaminants when making direct physical contact with the interior of a cannula device when moved back-and-forth following use of the cannula device, to be moved back and forth to cause frictional engagement with filter screen assembly 12 for cleaning. The cannula cleaner may be made of medical-grade nylon in some embodiments. In some embodiments, the cannula cleaner may be configured as shown and described in U.S. Provisional Application No. 62/855,167 entitled “Method and Apparatus for Cleaning the Interior Cannula of Suction Lipoplasty Cannula Devices and Adipose Tissue and/or Fluid Particle Sizing Devices,” filed on May 31, 2019, which is hereby incorporated by reference.

Adipose tissue particle processing system 10 described herein allows adipose tissue material to be micro-fragmented (“sized”) to a controllable fat aspirate particle size, with easy connections of components, in a system that minimizes contamination, spillage, and infection issues, while maintaining an essentially closed system during the processing of tissue and/or fluid.

While various components of adipose tissue particle processing system 10 are shown and/or described in the exemplary embodiments herein as integrated, connected, or separate components, it should be understood that in alternative embodiments, components may be integrally formed, connected, and/or separated in different ways than are shown and described herein, all within the scope and spirit of the present invention. Similarly, the sizes and dimensions of components, both in terms of absolute sizes and relative sizes with respect to other components, may be varied from what is shown and described herein, all within the scope of the present invention.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. An adipose tissue particle processing system comprising:
a container; and
a filter screen assembly at least partially extending into an interior of the container, the filter screen assembly comprising:
a first open end configured to receive adipose tissue material from a syringe;
a second closed end opposite to the first open end, the second closed end being located in the interior of the container; and
a screen portion between the first open end and the second closed end, the screen portion including a plurality of apertures having diameters selected for processing the adipose tissue material received through the first open end into controlled fat aspirate particle sizes that are output through the plurality of apertures into the interior of the container.
